(19) United States
(12) Patent Application Publication
(10) Pub. No.: US 2004/0101493 A1
(43) Pub. Date: May 27, 2004

(54) Chewable solid unit dosage forms and methods for delivery of active agents into occlusal surfaces of teeth

(76) Inventors: Douglas Craig Scott, Loveland, OH (US); Sandra Lynn Eversole, Maineville, OH (US); Steven Carl Burgess, Sharonville, OH (US); John Michael Best, Fairfield, OH (US); Robert Vincent Faller, Loveland, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/706,103

(22) Filed: Nov. 12, 2003

Related U.S. Application Data
(60) Provisional application No. 60/429,234, filed on Nov. 26, 2002.

(51) Int. Cl. A61K 7/16

(52) U.S. Cl. 424/49

(57) ABSTRACT

The present invention relates to methods and an oral care composition for topical, oral administration in a human or other animal comprising: a. from about 1% to about 40%, by weight of the composition, of a retentive agent selected from the group consisting of water soluble hydrophilic gums, water soluble hydrophilic polymers, and mixtures thereof, the retentive agent having the property of hydrating upon exposure to water or saliva resulting in the composition forming an intact hydrated mass to provide a Retention Index of about 1 to about 4; and b. a safe and effective amount of a topical, oral care carrier; wherein the composition is a non-cariogenic, chewable solid unit dosage form; and the composition comprises less than about 65% by weight of water insoluble particulates.

The present invention further relates to an oral care dentifrice composition comprising: a. from about 30% to about 65%, by weight of the composition, of a water insoluble, particulate retentive agent having a water solubility of less than about 1 g/30 g at 25° C.; b. a safe and effective amount of an oral care active; c. a safe and effective amount of a surfactant; d. a safe and effective amount of a buffer; wherein the composition is a chewable dentifrice solid unit dosage form, is non-effervescent, non-cariogenic; and wherein the composition has a Retention Index of from about 1 to about 4.

### CROSS REFERENCE TO RELATED APPLICATION

This application claims the benefit under 35 USC 119(e) to U.S. Application No. 60/429,234, filed Nov. 26, 2002.

### TECHNICAL FIELD

This invention relates to solid, chewable unit dosage form compositions and methods for delivery (especially sustained delivery) of fluoride or other oral care active agents in the oral cavity. The mechanical forces of biting or chewing by a subject are utilized to deposit and retain a minimum amount of the present composition into the tooth surfaces, especially the pits, fissures and occlusal surfaces of the teeth. The present compositions comprise a retentive agent and optionally one or more agents such as an oral care active agent, abrasive, foaming agent, flavors/sensates, and/or a specific buffer system. This invention also relates to solid, chewable compositions and methods that provide pH buffering on or at the tooth surfaces and in the oral cavity. These compositions include chewable dentifrice tablets.

### BACKGROUND ART

Many attempts have been made to control or prevent both the occurrence of caries and the formation of dental plaque. For example fluoride solutions or gels are used and are typically applied in the dentist office at periodic, but infrequent, intervals. Dental plaque results when cariogenic bacteria such as Streptococcus mutans collect in colonies and form deposits on tooth surfaces. The presence of bacteria and deposits is damaging to the teeth and gums and can lead to gingivitis, caries, periodontal disease and tooth loss.

The prior art teaches a variety of agents useful to alter the progression of variety of oral care conditions including agents that provide caries, antimicrobial, anticalculus, anesthetic, whitening, and/or anti-inflammatory efficacy. In particular it has long been known that fluoride-providing compounds are a safe and effective means for the promotion of the remineralization process.

In addition, the prior art teaches the use of tablet dosage forms for various oral care utilities. For example tooth cleaning tablets are disclosed in U.S. Pat. No. 4,753,792, issued Jun. 28, 1988. Specifically this reference teaches a tooth cleaning tablet which is self-foaming and self-cleansing on chewing and which includes a self-foaming effervescent couple composition which enables the tablet to readily form a foam on chewing without the need for agitation with a toothbrush. Furthermore, U.S. Pat. No. 3,962,417, Howell et al., teaches a tablet comprising approximately 70-75% by weight, acid neutralizer and approximately 17-20%, by weight, acid. The initial reaction of the acid neutralizer and the acid serves to create an effervescent action in the mouth, and the resulting basic solution then neutralizes the acidic Bacillus Acidophilios. U.S. Pat. No. 5,496,541, issued Mar. 5, 1996, teaches dental products, which can be in tablet form, employing a ternary surfactant system of poloxamers, anionic polysaccharides, and nonionic cellulose ethers for greatly enhanced foaming power.

Despite the above known prior art and technologies for treatment of oral conditions, the prior art has not fully appreciated the benefits of, or solved problems associated with, the delivery of oral care active agents directly into the tooth surfaces such as the pits, fissures or occlusal surfaces of the teeth with chewable solid unit dosage forms. The present invention provides these benefits through the mechanical shear provided by biting or chewing the solid unit dosage form and through the use of a retentive agent. The retentive agent enhances deposition and adhesion of the composition to the teeth surfaces. Also the prior art has not suggested an adequate means to provide pH buffering on or at the tooth surfaces, especially the sites where most caries form, the pits, fissures and occlusal surfaces of the teeth. These benefits are achieved, for example, through the selection of the ingredients and the levels of the components of the present invention.

### SUMMARY OF THE INVENTION

The present invention relates to an oral care composition for topical, oral administration in a human or other animal comprising:

a. from about 1% to about 40%, by weight of the composition, of a retentive agent selected from the group consisting of water soluble hydrophilic gums, water soluble hydrophilic polymers, and mixtures thereof, the retentive agent having the property of hydrating upon exposure to water or saliva resulting in the composition forming an intact hydrated mass to provide a Retention Index of about 1 to about 4; and

b. a safe and effective amount of a topical, oral care carrier; wherein the composition is a non-cariogenic, chewable solid unit dosage form; and the composition comprises less than about 65% by weight of water insoluble particulates.

The present invention further relates to an oral care dentifrice composition comprising:

a. from about 30% to about 65%, by weight of the composition, of a water insoluble, particulate retentive agent having a water solubility of less than about 1 g/30 g at 25° C.;

b. a safe and effective amount of an oral care active;

c. a safe and effective amount of a surfactant;

d. a safe and effective amount of a buffer;

wherein the composition is a chewable dentifrice solid unit dosage form, is non-effervescent, non-cariogenic; and wherein the composition has a Retention Index of from about 1 to about 4.

The present invention further relates to a method of buffering the oral cavity saliva or environment on or at the tooth surfaces of a human or animal subject in need thereof, to a pH from about 7 to about 12, for at least about 2 minutes, by administering the above compositions, including a buffer, topically to the oral cavity.

The present invention further relates to a method of providing sustained delivery of an oral care active, flavor, sensate or buffer, in the oral cavity of a human or animal subject in need thereof, by administering the above compositions topically to the oral cavity.

### BRIEF DESCRIPTION OF THE DRAWINGS

The present invention will be better understood by reference to the following detailed description of embodiments in conjunction with the accompanying drawings, in which like reference numerals identify identical elements. Without intending to limit the invention, embodiments of the present invention are described in more detail below.

FIG. 1 FIG. 1 is a photograph of a human subject's molar, taken at approximately 5, 15, 30, 45, and 60 minutes, respectively, after the subject chews a compressed tablet of the present invention and thereafter brushes the teeth, expectorates, and rinses the oral cavity with water.

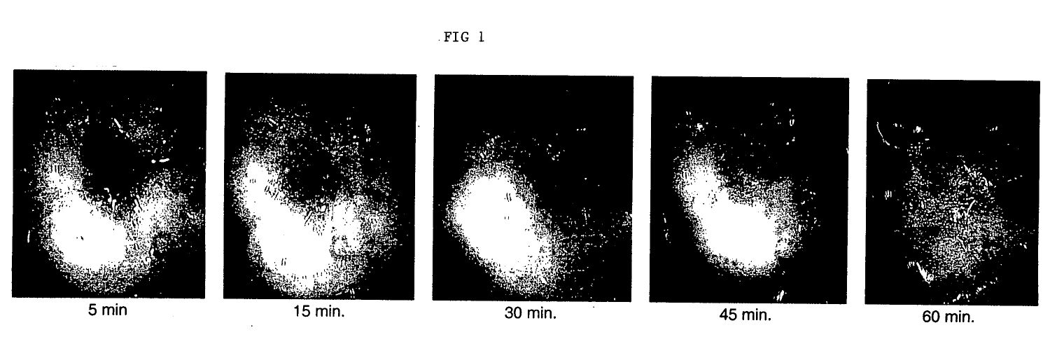

FIG. 2 FIG. 2 is a diagram of a human subject's full set of teeth, the red color showing the location of deposited tablet material after the subject uses the present invention. The photograph directly below each diagram corresponds to a partial view of two actual molars having tablet material deposited therein. The diagram and photographs are taken at approximately 5, 15, 30, 45, and 60 minutes, respectively, after the subject chews a compressed tablet of the present invention and thereafter brushes the teeth, expectorates, and rinses the oral cavity with water.

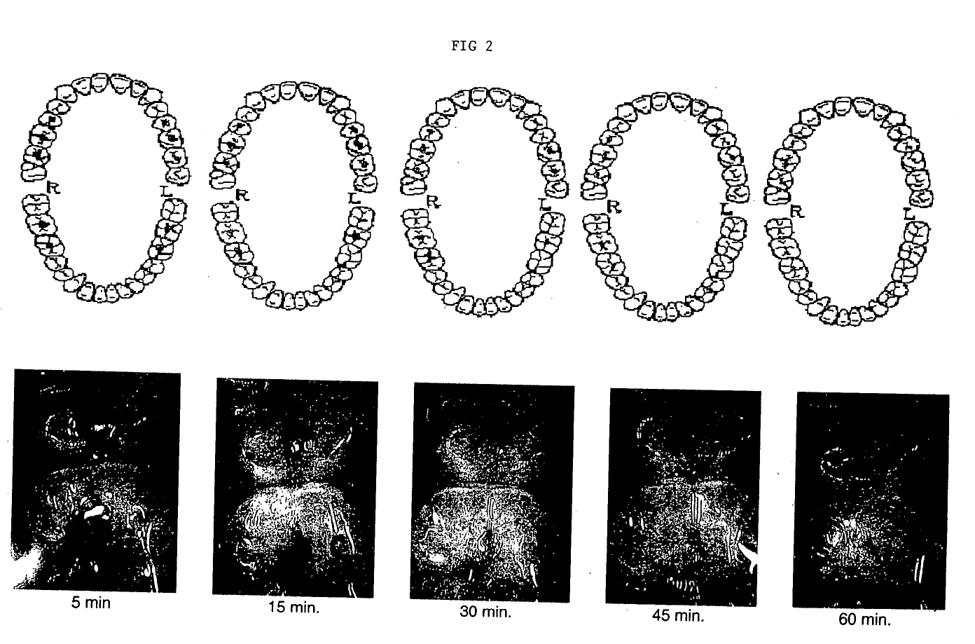

FIG. 3 FIG. 3 is a diagram of a human subject's full set of teeth, the red color showing the location of deposited tablet material after the subject uses the present invention. The photograph directly below each diagram corresponds to a partial view of one actual molar having tablet material deposited therein. The diagram and photographs are taken at approximately 5, 15, 30, 45, and 60 minutes, respectively, after the subject chews a compressed tablet of the present invention and thereafter brushes the teeth, expectorates, and rinses the oral cavity with water.

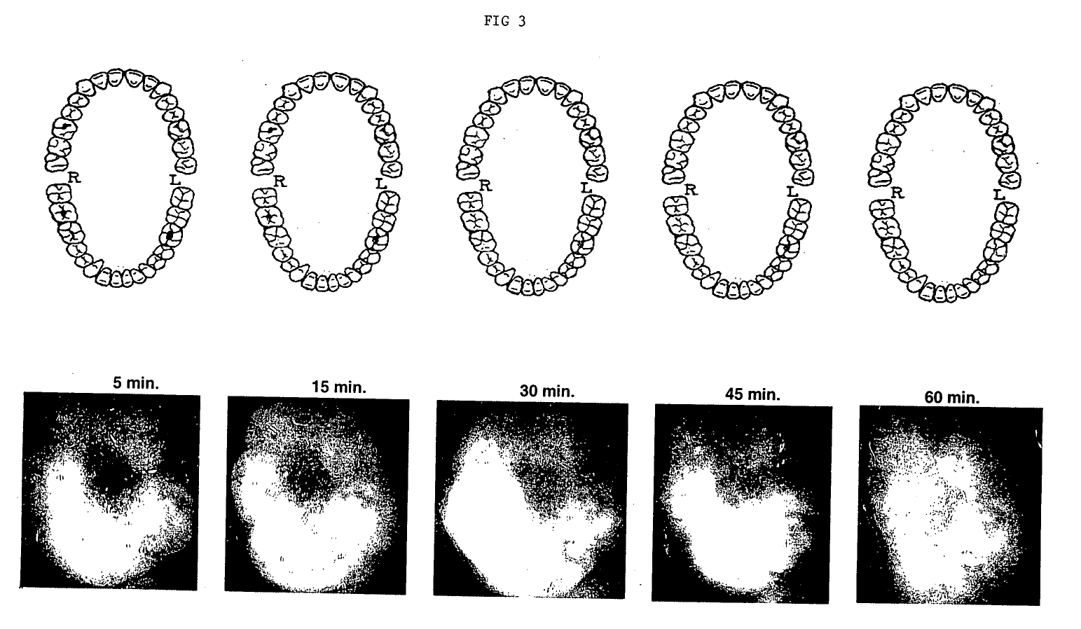

### DETAILED DESCRIPTION

Definitions

By “natural dentition” as used herein, means human subjects having natural teeth, the subjects having no more than one or two restorations or filings in their teeth, in another embodiment no more than three restorations or filings, and having at least 8 molars (including premolars). In addition, the subjects do not have sealants or veneers on their teeth and their teeth have normal morphology, e.g. lack relatively flat surfaces on their molars. Grinding of the teeth can cause relatively flat molar surfaces where the cusp tips become flattened. Restorations include crowns and filings.

By “oral care composition” or “oral composition” as used herein is meant a product which is not intentionally swallowed for purposes of systemic administration of therapeutic agents, but is retained in the oral cavity for a sufficient time to contact some or substantially all of the dental surfaces and/or oral mucosal tissues for purposes of oral activity. In addition these terms can mean a product which may be intentionally swallowed but not swallowed for the purposes of systemic administration of therapeutic agents.

By “oral condition” as used herein is meant diseases or conditions of the oral cavity including caries, plaque, breath malodor, dental erosion, gingivitis, and periodontal disease. Oral conditions are further described in WO 02/02096A2, published Jan. 10, 2002, P&G.

By “safe and effective amount” as used herein is meant an amount of a component, high enough to significantly (positively) modify the condition to be treated or to effect the desired anticaries result, but low enough to avoid serious side effects (at a reasonable benefit/risk ratio), within the scope of sound medical/dental judgment. The safe and effective amount of a component, will vary with the particular condition (e.g., to effect anticaries activity or remineralization effect) being treated, the age and physical condition of the patient being treated, the severity of the condition, the duration of treatment, the nature of concurrent therapy, the specific form employed, and the particular vehicle from which the component is applied.

By “toothpaste” as used herein is meant a product which is not intentionally swallowed for purposes of systemic administration of therapeutic agents, but is retained in the oral cavity for a sufficient time to contact some or substantially all of the dental surfaces and/or oral mucosal tissues for purposes of oral activity, unless otherwise specified.

By “tooth surfaces” or “teeth surfaces” as used herein is meant the pits, fissures, occlusal surfaces, cleft, crevices, grooves, depressions, interstices, irregularities, inter-proximal surfaces between the teeth and/or along the gum line, the smooth surfaces of teeth, and/or the grinding or biting surfaces of a tooth.

Herein, “comprising” means that other steps and other ingredients which do not affect the end result can be added. This term encompasses the terms “consisting of” and “consisting essentially of”.

By “whole body health” as used herein is meant overall systemic health characterized by a reduction in risk of development of major systemic diseases and conditions including cardiovascular disease, stroke, diabetes, severe respiratory infections, premature births and low birth weights (including post-partum dysfunction in neurologic/development function), and associated increased risk of mortality. It is believed that oral infections could lead to systemic infection. Bacteria can spread from the mouth into the bloodstream and other parts of the body, thereby putting a person's health at risk. Oral infection may contribute to the development of a number of serious conditions including heart disease, diabetes, respiratory diseases and premature, underweight births. Whole body health and promotion thereof by treating oral cavity infections is further described in WO 02/02063A2, WO 02/02096A2, WO 02/02128A2, all published Jan. 10, 2002.

All percentages and ratios used hereinafter are by weight of total composition, unless otherwise indicated.

All measurements referred to herein are made at 25° C. unless otherwise specified.

All percentages, ratios, and levels of ingredients referred to herein are based on the actual amount of the ingredient, and do not include solvents, fillers, or other materials with which the ingredient may be combined as a commercially available product, unless otherwise indicated.

All publications, patent applications, and issued patents mentioned herein are hereby incorporated in their entirety by reference. Citation of any reference is not an admission regarding any determination as to its availability as prior art to the claimed invention.

### Retentive Agent

An essential ingredient of the present invention is a safe and effective amount of a retentive agent. The retentive agent functions to allow at least a minimum amount of the composition to pack on some of the tooth surfaces for a minimum period of time after the subject bites or chews the solid unit dosage form (or after the subject bites or chews and thereafter brushes the teeth with the solid unit dosage form). The mechanical forces of biting or chewing aids to pack and deposit some of the dosage form on the tooth surfaces, especially the pits and fissures. These compositions, through the mechanical force of biting or chewing, pack or conform to the topography of some of the tooth surfaces, and thus may provide a temporary physical barrier or seal to protect the tooth surface from bacteria, acids, food, staining materials, and other material, as well as may provide extended delivery of oral care active agents directly to the tooth surfaces or in the oral cavity. The retentive agent must have sufficient binding properties to adhere to the tooth surface chemically and/or physically. In one embodiment, for toothpaste solid unit dosage forms, the retentive agent should provide an aesthetically pleasing viscous slurry formed during use from the portion of the dosage form which is not packed or deposited on the tooth surfaces. In one embodiment the retentive agent should not provide a negative feeling or presence in the mouth, e.g. not too sticky, gummy, slimy, etc.

In one embodiment the composition and methods herein has an average Retention Index (herein “RI”) of about 1 to about 4, in another embodiment from about 2 to about 4. The RI is calculated as follows. First, at least about 5 human subjects (in one embodiment at least about 10, and in another embodiment at least about 20 subjects), having natural dentition are selected. These subjects chew two tablets (one tablet on each side of the mouth) for about 5 seconds to about 30 seconds. Thereafter the subjects brush his/her teeth with a manual, flat head, soft toothbrush for about 30 seconds (in another embodiment for about 1 minute). The subjects thereafter expectorate the slurry created from the brushing. Then, the subjects optionally rinse with about 10 mils of water and expectorate again. After five minutes (in another embodiment after about 8 minutes and in another embodiment after about 10 minutes) all surfaces of the subject's teeth are graded visually based on the following scale:

| | | Number of | |
|---|---|---|---|
| | | Molar/Premolar | |
| | | Surfaces Having | Total Time Deposited |
| Retention | Amount of Deposited | Deposited | Material Remains |
| Index | Material | Material | Visable |
| 0 | None | 0 | 0 |
| 1 | Enough to be Visable | 2-3 surfaces | From about 1 minute to about 60 minutes; |
| | | | in another embodiment from about 10 |
| | | | minutes to about 35 minutes |
| 2 | Enough to be Visable | 4-5 surfaces | From about 1 minute to about 60 minutes; |
| | | | in another embodiment from about 10 |
| | | | minutes to about 35 minutes |
| 3 | Enough to be Visable | 6-7 surfaces | From about 1 minute to about 60 minutes; |
| | | | in another embodiment from about 10 |
| | | | minutes to about 35 minutes |
| 4 | Enough to be Visable | Greater than 7 | From about 1 minute to about 60 minutes; |
| | | surfaces | in another embodiment from about 10 |
| | | | minutes to about 35 minutes |

If a subject has material deposited on separate surfaces of a single tooth, e.g. at the gumline and in the pit of a molar, those surfaces are counted separately. “Visable” herein means that at least enough material is deposited to be seen by the naked eye.

For purposes of measuring the RI, for white colored tablets or tablets that have the same or similar color as that of the subjects teeth, after the subject expectorates the slurry, the subject rinses with from 5 to 10 mils of a water solution also containing a dye or contrast agent. The deposited material will thereafter have a contrasting color versus the color of the teeth. It is to be noted, however, the solid unit dosage forms herein can be any color or shape.

In one embodiment, after chewing by the subject (and optionally after brushing), from about 0.5% to about 20%, by weight of the initial composition, is deposited on some of the surfaces of the teeth, in another embodiment from about 0.8% to about 15% by weight, in another embodiment from about 1% to about 10% by weight, and in even another embodiment from about 1% to about 5% by weight of the initial composition. Once deposited on the teeth, some of the composition remains adhered to the surface of some of the teeth for at least about 2 minutes, in another embodiment for at least about 5 minutes, in another embodiment for at least about 10 minutes, in another embodiment for about 1 minute to about 1 hour, in another embodiment from about 10 minutes to about 35 minutes and in yet another embodiment from about 15 to about 30 minutes.

In one embodiment the retentive agent is a hydrophilic water soluble, gum or polymeric material that will form a hydrated mass upon hydration with aqueous fluids (water or saliva). In one embodiment formation of a gel occurs in about 1 to about 120 seconds, in another embodiment from about 5 to about 60 seconds, after exposure to water or saliva. Adequate speed of hydration will minimize the dissolution, disintegration, or erosion of the material deposited in the tooth surfaces, as well as minimize further rapid penetration of water or saliva into the deposited material. In one embodiment the present composition comprises from about 1% to about 40%, in another embodiment from about 2% to about 40%, in another embodiment from about 7% to about 25%, in another embodiment from about 8% to about 20%, and in even another embodiment from about 11% to about 18%, by weight of the composition of the hydrophilic water soluble, gum or polymeric, retentive agent.

In one embodiment the retentive agent is selected from the group consisting of acacia, karaya gum, guar gum, gelatin, alginic acid and salts thereof (e.g. sodium alginate), polyethylene glycol, polyethylene oxide, acrylamide polymers, cross linked polyacrylic acid, hydrophobically modified polyacrylic acid polymers, polyvinyl alcohol, ethylene oxide polymers, polyvinylpyrrolidone, cationic polyacrylamide polymers, cellulose derivatives such as carboxymethylcellulose, hydroxyethylcellulose, hydroxypropylcellulose, hydroxypropylmethylcellulose; xanthan gum, carrageenan, locust bean gum, gum Arabic, tragacanth gum, pullulan, pre-gelatinized and partially pre-gelatinized starch, hydrolyzed starch, maltodextrin and corn syrup solids, hydrogenated maltodextrin, hydrogenated starch hydrosylates, amylose, amylopectin, starch derivatives, and mixtures thereof.

In another embodiment the retentive agent is selected from the group consisting of acacia, karaya gum, guar gum, gelatin, alginic acid and salts thereof (e.g. sodium alginate), polyethylene oxide, acrylamide polymers, cross linked polyacrylic acid, polyvinyl alcohol, cationic polyacrylamide polymers, carboxymethylcellulose, hydroxyethylcellulose, hydroxypropylcellulose, hydroxy-propylmethylcellulose, xanthan gum, carrageenan, locust bean gum, gum Arabic, tragacanth gum, pullulan, pre-gelatinized and partially pre-gelatinized starch, hydrolyzed starch, maltodextrin and corn syrup solids, hydrogenated starch hydrosylates, amylose, amylopectin, starch derivatives, and mixtures thereof.

In another embodiment the retentive agent is selected from the group consisting of acacia, karaya gum, guar gum, alginic acid and salts thereof (e.g. sodium alginate), carboxymethylcellulose, hydroxyethylcellulose, hydroxypropylcellulose, hydroxy-propylmethylcellulose, carrageenan, locust bean gum, gum Arabic, tragacanth gum, pullulan, and mixtures thereof.

In another embodiment the retentive agent is selected from the group consisting of hydroxy-propylmethylcellulose (HPMC), hydroxyproplycellulose, carboxymethylcellulose, hydroxyethyl cellulose, and mixtures thereof.

In one embodiment the retentive agent is a relatively hydrophilic polymer or gum, e.g. having a higher relative level of hydrophilic group substitution (e.g. from about 7% to about 12% hydroxypropyl substitution) and a lower relative level of hydrophobic substitution (e.g. from about 19% to about 24% methoxyl substitution), such as Methocel K (hydroxypropyl methylcellulose type 2208 from Dow Chemical Co.), Methocel K4M Premium, and K100LV Premium grades (Dow Chemical Company), etc.

In one embodiment the retentive agent is a hydrophilic polymer or gum having a relatively small particle size, for example at least 75% of the polymer passes through a 200 mesh sieve, in another embodiment at least 75% of the polymer passes through a 100 mesh sieve, such as Methocel K (hydroxypropyl methylcellulose type 2208 from Dow Chemical Co.), cellulose polymers which have a high level of hydroxypropyl substitution and a low level of methoxyl substitution, Methocel K4M Premium and K100LV Premium grades (Dow Chemical Company), etc.

In one embodiment the retentive agent is a mixture of Methocel K4M Premium and K100LV Premium grades (Dow Chemical Company) at a ratio of from about 1:1 to about 1:2.5, Methocel K4M Premium to Methocel K100LV Premium.

In another embodiment the retentive agent is Methocel E (hydroxypropyl methylcellulose type 2910 from Dow Chemical Co.), which has a level of hydroxypropyl substitution of 7-12% and a level of methoxyl substitution of 28-30%.

In one embodiment the composition comprises from about 1% to about 20% by weight, in another embodiment from about 1% to about 18% by weight, and in another embodiment is from about 3% to about 16% by weight, of a retentive agent that is a hydrophilic water soluble gum or polymeric material having a viscosity of from about 80 cps to about 20,000 cps, in another embodiment from about 100 cps to about 15,000 cps and in yet another embodiment is from about 150 cps to about 10,000 cps. These viscosities are determined by the method provided in the USP Official Monographs for hydroxypropyl methylcellulose and physical tests for viscosity. In one embodiment these lower viscosity materials are mixed with a higher viscosity hydrogel material (e.g. with viscosities of from about 21,000 cps to about 100,000 cps).

In one embodiment the retentive agent is Natrasol 250 available from Aqualon, or medium or higher viscosity hydroxyethyl cellulose available from Aqualon.

In one embodiment the retentive agent is a high viscosity carboxymethylcellulose such as Carboxymethylcellulose 7H3, available from Aqualon having an average viscosity of about 3,000 cps, Carboxymethylcellulose 9H4, available from Aqualon having an average viscosity of about 4,000 cps, and Aquasorb A 500 available from Aqualon.

In one embodiment the retentive agent includes a class of homopolymers of acrylic acid crosslinked with an alkyl ether of pentaerythritol or an alkyl ether of sucrose, or carbomers. Carbomers are commercially available from B.F. Goodrich as the Carbopol® series. Particularly preferred carbopols include Carbopol 934, 940, 941, 956, and mixtures thereof.

Specific sources of the above retentive agents are as follows. Acacia, guar gum, tragacanth, xanthan gum, locust bean gum, guar gum, and agar are available in various grades from Gumix International. Carrageenan and pectin are available under the tradename Genu® from Kelco; karaya gum (Keltrol® from Kelco); konjac (FMC); gelatin (Kind and Knox); alginic acid and salts thereof, e.g. sodium alginate and propylene glycol alginate (Protanol® FMC and Kelcoid/Kelgin® Kelco); polyethylene glycol (Carbowax® Union Carbide); ethylene oxide polymers, polyethylene oxide (Polyox® Union Carbide), polyvinyl alcohol (Elvanol® Du Pont); polyvinylpyrrolidone and derivatives (Plasdone®, ISP; Kollidone® BASF); cross linked polyacrylic acids, salts and derivatives thereof (Carbopol® Noveon, and Polycarbophil®, BF Goodrich/Noveon; hydrophobically modified polyacrylic acid polymers (sold as Carbopol® 1342 and 1382, and Carbopol® ETD 2020, and Pemulen® TR-1, TR-2, 1621, and 1622, all available from BF Goodrich), carboxymethylcellulose (Cekol® Metsa-Serla; hydroxyethylcellulose (Natrosol® Aqualon®/Hercules); hydroxypropylcellulose (Klucel® Aqualon®/Hercules); hydroxy-propylmethylcellulose (Methocel® Dow); pre-gelatinized and partially pre-gelatinized starch (Unipure®/National 78-1551, National Starch; Starch 1500, Colorcon); hydrolyzed starch, maltodextrin and corn syrup solids (Maltrin® Grain Processing); hydrogenated starch hydrosylates (Hystar® SPI Polyols).

In another embodiment the retentive agent may be a water insoluble particulate retentive agent having a water solubility of less than about 1 g/30 g at 25° C., in another embodiment less than about 1 g/100 g at 25° C., in yet another embodiment less than about 1 g/1000 g at 25° C. The level of the particulate retentive agent is generally less than about 65% by weight, in another embodiment less than about 60%, and in another embodiment is from about 30% to about 65%, in another embodiment is from about 30% to about 60%, and in another embodiment is from about 35% to about 55%, by weight of the composition. Examples of particulate retentive agents include calcium carbonate, mica, titanated mica, magnesium carbonate, talc (magnesium silicate), magnesium aluminum silicate, kaolin (aluminum silicate), titanium dioxide, zinc oxide, polyethylene powder, polystyrene powder, bismuth oxychloride, and mixtures thereof.

In one embodiment the particulate retentive agent is selected from the group consisting of calcium carbonate, magnesium carbonate, talc (magnesium silicate), magnesium aluminum silicate, and mixtures thereof.

In one embodiment the compositions of the present invention have less that about 5% by weight, in another embodiment less than about 2% by weight, and in yet another embodiment are essentially free of, starches, sugars, polysaccharides or fermentable sugars, that are known to be cariogenic (e.g. sucrose, etc.). The possible cariogenic effects that may result from the use of the above listed starches as retentive agents may be counteracted by the inclusion of fluoride ions, buffers and/or the use of non-cariogenic polysaccharides, in the present compositions.

In one embodiment the present compositions are not effervescent compositions. In one embodiment the retentive agent is noncariogenic.

In one embodiment these compositions have less than about 65%, in another embodiment less than about 60%, and in another embodiment less than about 55%, of water insoluble particulates (for example dental abrasives or other particulate carriers, etc.) having a water solubility of less than about 1 g/30 g at 25° C., in another embodiment less than about 1 g/100 g at 25° C., in yet another embodiment less than about 1 g/1000 g at 25° C.

Retention Modifiers

In one embodiment, to increase or decrease the retention properties of the composition, the composition can optionally comprise retention modifiers at a level from about 0.5% to about 20%, in another embodiment from about 2% to about 18%, in another embodiment from about 2% to about 15%, by weight of the composition. These retention modifiers are selected from the group consisting of bentonites, pectin, fats, waxes, shellac, ethyl cellulose, insoluble polymers, surfactants, clays, zein, cyclodextrins (Kleptose, Roquette); proteins and hydrolyzed protein (e.g. Crotein® from Croda), alkyl vinyl ether-maleic acid or anhydride copolymer and salts thereof, and mixtures thereof. In addition these retention modifiers may add hydrophobicity to the solid unit dosage form to slow down erosion or dissolution of the active agent from the deposited material. Alkyl vinyl ether-maleic acid or anhydride copolymers are employed in the form of their free acids or partially or fully neutralized alkali metal salts (e.g. zinc, magnesium, iron, calcium, strontium, potassium, and sodium) or ammonium salts, and mixtures thereof, and are disclosed in U.S. Pat. No. 6,475,498, Rajaiah et al., issued Nov. 2, 2002; U.S. Pat. No. 6,475,497, Rajaiah et al, issued Nov. 2, 2002, and include Gantrez AN 139 (M.W. 500,000), A.N. 119 (M.W. 250,000), AN 169, and S-97 Pharmaceutical Grade (M.W. 70,000), of GAF Corporation.

### Optional Buffering Agent and pH

The present compositions may optionally include a buffer. In one embodiment the present invention relates to a composition and method whereby the saliva or the environment on or at the tooth surfaces is buffered to a pH of from about 7 to about 12. This buffering action of the chewable sold unit dosage forms of the present invention may provide improved efficacy against the formation of caries lesion in the oral cavity. Improved anticaries efficacy may be achieved by directly neutralizing the acid environment existing on or at the tooth surfaces, especially the pits, fissures or occlusal tooth surfaces where most caries form.

Any suitable buffer may be selected for use herein at a safe and effective amount. In one embodiment the buffer may be selected from the group consisting of water soluble buffers such as sodium bicarbonate, sodium carbonate, phosphate buffers, amino acid buffers such as alanine and glycine, and mixtures thereof. In another embodiment the buffer is selected from the group consisting of sodium bicarbonate, sodium carbonate, trisodium phosphate, disodium phosphate, disodium hydrogen phosphate, sodium dihydrogen phosphate, tris(hydroxymethyl)aminomethane, tetrasodium pyrophosphate, disodium pyrophosphate; tetrapotassium pyrophosphate, salts of tripolyphosphates, and mixtures thereof. In another embodiment the buffer is sodium bicarbonate, sodium carbonate and mixtures thereof. The buffer can also include a water insoluble buffering agent for example, calcium carbonate.

In one embodiment the present composition comprises from about 0.1% to about 25%, in another embodiment from about 1 to about 20%, and in another embodiment from about 5 to about 18%, by weight of the composition of a buffer.

Disodium phosphate is also known as disodium orthophosphate, dibasic sodium phosphate, phosphate of soda, and secondary sodium phosphate.

After chewing a composition herein comprising a buffer, the pH of the saliva and/or the environment on or at the tooth surface, is from about 7 to about 12, in another embodiment from about 7.5 to about 10, in another embodiment from about 8 to about 9. As used herein the “the environment on or at the tooth surface” means the tooth surface that is adjacent to the solid unit dosage form impacted or deposited on the tooth surface and is not directly touching the material that is impacted on the tooth surface. This pH is sustained for at least about 2 minutes, in another embodiment for at least about 5 minutes, in another embodiment for at least about 15 minutes and in yet another embodiment for at least about 30 minutes. In another embodiment this pH is sustained from about 5 minutes to about 60 minutes, in another embodiment from about 5 minutes to about 30 minutes.

The pH may be measured by the following procedure. The subject, with natural dentition, chews a unit dosage form of the present invention until the unit dosage form is broken up (e.g. chews for about 5 seconds to about 30 seconds). Optionally, thereafter the subject brushes his/her teeth for about 30 seconds, in another embodiment for about 1 minute, with a manual, flat head soft toothbrush. The subject thereafter expectorates and optionally rinses with about 10 mls of water and expectorates again. Saliva is collected using a sponge tipped Critical swab. The sponge tip is placed on the environment on or at the tooth surface. The swab handle is then cut to an approximate length of 1.5 mm and then placed in a micro-centrifuge tube (swab end up). Samples are centrifuged for 10 minutes at 10,000 rpm. Swabs are removed from tubes leaving only saliva remaining. The pH of this saliva is measured using a micro pH electrode (e.g. Thermo Orion micro combination #9810BN) connected to a Corning pH meter Model 430. A pH measurement may be taken at various time periods after chewing and deposition, i.e. at 5, 10, 15, 30, minutes.

Alternatively, a saliva sample (2-5 mils) is removed from the oral cavity and the pH of the saliva sample is measured by any appropriate pH electrode.

### Optional Oral Care Active Agent

The present invention may optionally comprise a safe and effective amount of an oral care active agent selected from the group consisting of anticalculus agent, fluoride ion source, antimicrobial agents, dentinal desensitizing agents, anesthethic agents, antifungal agents, anti-inflammatory agents, selective H-2 antagonists, anticaries agents, remineralization agents, whitening agents, antierosion agents, vitamins and minerals, and mixtures thereof and in another embodiment selected from the group consisting of anticalculus agent, fluoride ion source, antimicrobial agents, anticaries agents, and mixtures thereof. These oral care active agents are useful for treating one or more oral conditions.

The oral care active agents can be present in the solid dosage forms in suitable unit dosage amounts. These amounts will be known by those skilled in the art and are disclosed below.

In one embodiment an advantage of the chewable unit dosage forms of the present invention is that the composition may provide efficacy at lower doses of oral care active agents than those doses conventionally known and used in the prior art. Lower than conventional dosages may provide efficacy since the dosage of the oral care active agent is delivered directly to, and retained on, the tooth surfaces.

Anticaries Agents and Fluoride Ion Source

The present composition may optionally comprise a safe and effective amount of an anticaries agent, remineralization agent, and mixtures thereof. In one embodiment the anticaries agent is selected from the group consisting of xylitol, fluoride ion source, and mixtures thereof. The fluoride ion source provides free fluoride ions during the chewing of the composition. In one embodiment the oral care active agent is a fluoride ion source selected from the group consisting of sodium fluoride, stannous fluoride, indium fluoride, organic fluorides such as amine fluorides, and sodium monofluorophosphate. Sodium fluoride is the fluoride ion in another embodiment. Norris et al., U.S. Pat. No. 2,946,725, issued Jul. 26, 1960, and Widder et al., U.S. Pat. No. 3,678,154 issued Jul. 18, 1972, disclose such fluoride salts as well as others that can be used as the fluoride ion source.

An advantage of the chewable unit dosage forms of the present invention is that the composition may provide efficacy at lower doses of the oral care active agent since the dosage of the oral care active agent is delivered directly to, and retained for sufficient time, on the tooth surfaces. For example lower dosages of fluoride may be used, thus possibly providing a safety advantage, by delivering the fluoride ion source directly onto the teeth surfaces and providing a means whereby the fluoride is adhered directly to the area where most caries form, especially the pits, fissures and occlusal surfaces of the teeth.

In one embodiment the level of fluoride ion source is from about 5 ppm to about 3500 ppm, in another embodiment from about 10 ppm to about 3000 ppm, and in another embodiment from about 50 ppm to about 2,800 ppm, and in another embodiment from about 100 ppm to about 2,000 ppm, and in another embodiment from about 300 ppm to about 1,500 ppm, and in even another embodiment from about 850 ppm to about 1,100 ppm or from about 200 ppm to about 300 ppm, of free fluoride ions. In one embodiment the tablet size may range from about 250 mg to about 1500 mg, in another embodiment from about 250 mg to about 1,000 mg, and in another embodiment from about 250 mg to about 500 mg.

In one embodiment where the proper oral care active dosage is provided by one tablet, the tablets may be scored wherein the subject divides the tablet in half and places ½ tablet on each side of mouth before chewing. In one embodiment where the proper dosage is provided by two tablets, then the subject can place 1 tablet on each side of the mouth before chewing. Alternatively, where the proper dosage is one tablet (twice a day), the subject can chew once tablet on one side of the mouth in the morning and another tablet on the other side of the mouth in the evening.

Remineralization Agents

Other optional anticaries agents include those agents that remineralize enamel and dentine. These remineralization agents prevent, treat and/or reverse the caries process. The optional remineralization agents are selected from the group consisting of a calcium ion source that is saliva soluble or becomes soluble with increased heat or with pH changes and/or a phosphate ion source; complexes of a fluoride ion source with an insoluble or soluble calcium ion source and amorphous forms thereof; complexes of a fluoride ion source with an insoluble or soluble phosphate ion source and amorphous forms thereof; fluoride ion source with an insoluble or soluble calcium and phosphate ion source and amorphous forms thereof; amorphous forms; dicalcium phosphate; hydroxapatite; nano-hydroxyapatite; a combination of strontium EDTA complex and a soluble fluoride ion source; casein glycomacropeptide, and mixtures thereof.

Combinations of calcium, phosphate and/or fluoride are disclosed in U.S. Pat. No. 5,037,639, issued Aug. 6, 1991 Tung, U.S. Pat. No. 6,000,341, issued Dec. 14, 1999, Tung, U.S. Pat. No. 5,258,167, issued Dec. 7, 1993 Tung, U.S. Pat. No. 6,303,104, issued Oct. 16, 2001, Winston et al., U.S. Pat. No. 6,159,449, issued Dec. 12, 2000, Winston et al., U.S. Pat. No. 6,159,448, issued Dec. 12, 2000, Winston et al., U.S. Pat. No. 6,036,944, issued Mar. 14, 2000, Winston et al., U.S. Pat. No. 5,895,641, issued Apr. 20, 1989, Usen et al., U.S. Pat. No. 5,866,102, issued Feb. 2, 1999, Winston et al, U.S. Pat. No. 5,858,333, issued Jan. 12, 1999, Winston et al., U.S. Pat. No. 5,833,957, issued Nov. 10, 1998, Winston et al., U.S. Pat. No. 5,817,296, issued Oct. 6, 1998, Winston et al., U.S. Pat. No. 5,614,175, issued Mar. 25, 1997, U.S. Pat. No. 5,605,675, issued Feb. 25, 1997, Usen et al., U.S. Pat. No. 5,571,502, issued Nov. 5, 1996, Winston et al., U.S. Pat. No. 6,120,754, issued Sept. 19, 2000, Lee et al., U.S. Pat. No. 6,214,321, issued Apr. 10, 2001, Lee et al.

Casein glycomacropeptides are disclosed in U.S. Pat. No. 5,853,704, issued Dec. 29, 1998, Zhang, et al., U.S. Pat. No. 6,207,138, issued Mar. 27, 2001, Zhang et al., U.S. Pat. No. 5,741,773, issued Apr. 21, 1998, Zhang et al., U.S. Pat. No. 4,992,420, issued Feb. 12, 1991, Nesser.

The remineralization agent can comprise a combination of strontium EDTA complex and a soluble fluoride ion source such as disclosed in U.S. Pat. No. 4,978,522, Barbera et al, issued Dec. 18, 1989.

Nanocrystalline hydroxyapatite, having average size of 0.5 and 200 nm, are disclosed in WO 00/03747, published Jan. 27, 2000, Dolci et al. The nanohydroxyapatite of the present invention may also include those disclosed in U.S. Pat. No. 5,833,959, issued Nov. 10, 1998, Sangi Co., Atsumi et al., which teaches a composition for use in dental tissues with hydroxyapatite having a particle size up to about 1.0 μm and generally in a range from about 0.05 μm to about 1.0 μm at a minimun of 0.1% by weight. In Atsumi et al, hydroxyapatite is also referred to as calcium teriary phosphate. Other hydroxyapatite materials useful herein include those described in U.S. Pat. No. 4,923,683, Sakuma et al, assigned to Sangi, issued May 8, 1990 and U.S. Pat. No. 5,135,396, Kuboki, assigned to Sangi, issued Aug. 4, 1992.

These remineralization agents are optionally used at a level of from about 0.1% to about 20%, in another embodiment from about 0.5% to about 5%, and in yet another embodiment from about 1% to about 3% by weight.

Biologic Anticaries Agents

The present invention may also optionally comprise a safe and effective amount of a biologic material, for example, a type of oral cavity bacteria that causes or contributes to the development of caries that has been modified to render it less damaging in the caries process. For example, streptococcus mutans is believed to be a principal pathogen in dental caries, a disease characterized by the dissolution of the mineral portion of the tooth caused by acid resulting from the interaction of bacteria on the tooth surface with carbohydrates. Modified bacteria include, for example, recombinant Streptococcus mutans strains characterized by a deficiency in lactic acid production and production of a recombinant alcohol dehydrogenase (ADH) as described, in U.S. Pat. No. 5,607,672, issued Mar. 4, 1997, Hillman. Some of these mutant strains have been isolated from Streptococcus mutans strain BHT-2(str) which are characterized by a single point mutation in the structural gene for the enzyme, L(+) lactate dehydrogenase, this enzyme being normally responsible for lactic acid production by this bacterium. See for example U.S. Pat. No. 4,133,875 issued Jan. 9, 1979, Hillman and U.S. Pat. No. 4,324,860, issued Apr. 13, 1982, Hillman. These recombinant S. mutans strains are suitable for use for preventing or treating dental caries.

Anticalculus Agents

The present compositions may optionally comprise a safe and effective amount of at least one anticalculus agent. This amount is generally from about 0.01% to about 40% by weight of the composition, in another embodiment is from about 0.1% to about 25%, and in yet another embodiment is from about 4.5% to about 20%, and in yet another embodiment is from about 5% to about 15%, by weight of the composition. An effective amount of the anticalculus agent is released from the solid unit dosage form. The anticalculus agent should also be essentially compatible with the other components of the composition.

The anticalculus agent is selected from the group consisting of polyphosphates and salts thereof; polyamino propane sulfonic acid (AMPS) and salts thereof; polyolefin sulfonates and salts thereof; polyvinyl phosphates and salts thereof; polyolefin phosphates and salts thereof; diphosphonates and salts thereof; phosphonoalkane carboxylic acid and salts thereof; polyphosphonates and salts thereof; polyvinyl phosphonates and salts thereof; polyolefin phosphonates and salts thereof; polypeptides; and mixtures thereof. In one embodiment, the salts are alkali metal salts. In another embodiment the anticalculus agent is selected from the group consisting of polyphosphates and salts thereof; diphosphonates and salts thereof; and mixtures thereof. In another embodiment the anticalculus agent is selected from the group consisting of pyrophosphate, polyphosphate, and mixtures thereof.

Polyphosphate

In one embodiment of the present invention, the anticalculus agent is a polyphosphate. A polyphosphate is generally understood to consist of two or more phosphate molecules arranged primarily in a linear configuration, although some cyclic derivatives may be present. Linear polyphosphates correspond to (X PO₃)ₙ where n is about 2 to about 125, wherein preferably n is greater than 4, and X is for example sodium, potassium, etc. For (X PO₃)ₙ when n is at least 3 the polyphosphates are glassy in character. Counterions for these phosphates may be the alkali metal, alkaline earth metal, ammonium, C₂-C₆ alkanolammonium and salt mixtures. Polyphosphates are generally employed as their wholly or partially neutralized water soluble alkali metal salts such as potassium, sodium, ammonium salts, and mixtures thereof. The inorganic polyphosphate salts include alkali metal (e.g. sodium) tripolyphosphate, tetrapolyphosphate, dialkyl metal (e.g. disodium) diacid, trialkyl metal (e.g. trisodium) monoacid, potassium hydrogen phosphate, sodium hydrogen phosphate, and alkali metal (e.g. sodium) hexametaphosphate, and mixtures thereof. Polyphosphates larger than tetrapolyphosphate usually occur as amorphous glassy materials. In one embodiment the polyphosphates are those manufactured by FMC Corporation which are commercially known as Sodaphos (n≈6), Hexaphos (n≈13), and Glass H (n≈21), and mixtures thereof. The present compositions will typically comprise from about 0.5% to about 20%, in one embodiment from about 4% to about 15%, in yet another embodiment from about 6% to about 12%, by weight of the composition of polyphosphate.

The phosphate sources are described in more detail in Kirk & Othmer, Encyclopedia of Chemical Technology, Fourth Edition, Volume 18, Wiley-Interscience Publishers (1996), pages 685-707, incorporated herein by reference in its entirety, including all references incorporated into Kirk & Othmer.

In one embodiment the polyphosphates are the linear “glassy” polyposphates having the formula:

XO(XPO₃)ₙX

wherein X is sodium or potassium; and n averages from about 6 to about 125.

In one embodiment, when n is at least 2 in either of the above polyphosphate formulas, the level of anticalculus agent is from about 0.5% to about 40%, in another embodiment is from about 2% to about 25%, and in even another embodiment is from about 5% to about 15%, by weight of the composition. Polyphosphates are disclosed in U.S. Pat. No. 4,913,895.

Pyrophosphate

The pyrophosphate salts useful in the present compositions include, alkali metal pyrophosphates, di-, tri-, and mono-potassium or sodium pyrophosphates, dialkali metal pyrophosphate salts, tetraalkali metal pyrophosphate salts, and mixtures thereof. In one embodiment the pyrophosphate salt is selected from the group consisting of trisodium pyrophosphate, disodium dihydrogen pyrophosphate (Na₂H₂P₂O₇), dipotassium pyrophosphate, tetrasodium pyrophosphate (Na₄P₂O₇), tetrapotassium pyrophosphate (K₄P₂O₇), and mixtures thereof. The pyrophosphate salts described in U.S. Pat. No. 4,515,772, issued May 7, 1985, and U.S. Pat. No. 4,885,155, issued Dec. 5, 1989, both to Parran et al. The pyrophosphate salts are described in more detail in Kirk & Othmer, Encyclopedia of Chemical Technology, Third Edition, Volume 17, Wiley-Interscience Publishers (1982), pages 685-707.

In one embodiment, the compositions of the present invention comprise tetrasodium pyrophosphate. Tetrasodium pyrophosphate may be the anhydrous salt form or the decahydrate form, or any other species stable in solid form in the present compositions. The salt is in its solid particle form, which may be its crystalline and/or amorphous state, with the particle size of the salt preferably being small enough to be aesthetically acceptable and readily soluble during use.

The level of pyrophosphate salt in the compositions of the present invention is any safe and effective amount, and is generally from about 1.5% to about 15%, in another embodiment from about 2% to about 10%, and yet in another embodiment from about 3% to about 8%, by weight of the composition.

Azacycloalkane-2,2-diphosphonic acids are disclosed in U.S. Pat. No. 3,941,772, issued Mar. 2, 1976, Ploger et al., assigned to Henkel and U.S. Pat. No. 3,988,443, issued Oct. 26, 1976, Ploger et al.

Optional agents to be used in place of or in combination with the pyrophosphate salt include such known materials as synthetic anionic polymers, including polyacrylates and copolymers of maleic anhydride or acid and methyl vinyl ether (e.g., Gantrez), as described, for example, in U.S. Pat. No. 4,627,977, to Gaffar et al. as well as, e.g., polyamino propoane sulfonic acid (AMPS), zinc citrate trihydrate, polyphosphates (e.g., tripolyphosphate; hexametaphosphate), diphosphonates (e.g., EHDP; AHP), polypeptides (such as polyaspartic and polyglutamic acids), and mixtures thereof.

Antierosion Agents

Dental erosion is a permanent loss of tooth substance from the surface by the action of chemicals, such as harsh abrasives and acids, as opposed to subsurface demineralization or caries caused by bacterial action. Dental erosion is a condition that does not involve plaque bacteria and is therefore distinct from dental caries, which is a disease caused by acids generated by plaque bacteria. Dental erosion may be caused by extrinsic or intrinsic factors.

Antierosion agents may include, but are not limited to, polymeric mineral surface-active agents selected from the group consisting of condensed phosphorylated polymers; polyphosphonates; polycarboxylates and carboxy-substituted polymers; copolymers of phosphate- or phosphonate-containing monomers or polymers with ethylenically unsaturated monomers, amino acids, or with other polymers selected from proteins, polypeptides, polysaccharides, poly(acrylate), poly(acrylamide), poly(methacrylate), poly(ethacrylate), poly(hydroxyalkylmethacrylate), poly(vinyl alcohol), poly(maleic anhydride), poly(maleate) poly(amide), poly(ethylene amine), poly(ethylene glycol), poly(propylene glycol), poly(vinyl acetate) or poly(vinyl benzyl chloride); and mixtures thereof. In one embodiment the antierosion agent is selected from the group consisting of polyphosphates where n=21 (described above), tripolyphosphate, and mixtures thereof. Also useful as antierosion agents are metal ions selected from stannous, zinc, copper, and mixtures thereof. Antierosion agents are further described in US 2003/0165442A1, published Sep. 4, 2003

Antimicrobial Agents

Antimicrobial antiplaque agents may also by optionally present in the present compositions. Such agents may include, but are not limited to, triclosan, 5-chloro-2-(2,4-dichlorophenoxy)-phenol, as described in The Merck Index, 11th ed. (1989), pp. 1529 (entry no. 9573) in U.S. Pat. No. 3,506,720, and in European Patent Application No. 0,251,591 of Beecham Group, PLC, published Jan. 7, 1988; chlorhexidine (Merck Index, no. 2090), alexidine (Merck Index, no. 222; hexetidine (Merck Index, no. 4624); sanguinarine (Merck Index, no. 8320); benzalkonium chloride (Merck Index, no. 1066); salicylanilide (Merck Index, no. 8299); domiphen bromide (Merck Index, no. 3411); cetylpyridinium chloride (CPC) (Merck Index, no. 2024; tetradecylpyridinium chloride (TPC); N-tetradecyl4-ethylpyridinium chloride (TDEPC); octenidine; delmopinol, octapinol, and other piperidino derivatives; effective antimicrobial amounts of essential oils and combinations thereof for example citral, geranial, and combinations of menthol, eucalyptol, thymol and methyl salicylate; antimicrobial metals and salts thereof for example those providing zinc ions, stannous ions, copper ions, and/or mixtures thereof; bisbiguanides, or phenolics; antibiotics such as augmentin, amoxicillin, tetracycline, doxycycline, minocycline, and metronidazole; and analogs and salts of the above antimicrobial antiplaque agents; anti-fungals such as those for the treatment of candida albicans. If present, these agents generally are present in a safe and effective amount for example from about 0.1% to about 5% by weight of the compositions of the present invention.

Antiinflammatory Agents

Anti-inflammatory agents may also be present in the oral compositions of the present invention. Such agents may include, but are not limited to, non-steroidal anti-inflammatory agents such as aspirin, ketorolac, flurbiprofen, ibuprofen, naproxen, indomethacin, aspirin, ketoprofen, piroxicam and meclofenamic acid, COX-2 inhibitors such as valdecoxib, celecoxib and rofecoxib, and mixtures thereof. If present, the anti-inflammatory agents generally comprise from about 0.001% to about 5% by weight of the compositions of the present invention. Ketorolac is described in U.S. Pat. No. 5,626,838, issued May 6, 1997.

H-2 Antagonists

The present invention may also comprise a safe and effective amount of a selective H-2 antagonist including compounds disclosed in U.S. Pat. No. 5,294,433, Singer et al., issued Mar. 15, 1994.

Whitening Agents

Teeth whitening actives that may be used in the oral care compositions of the present invention a safe and effective amount of a bleaching agent that include bleaching or oxidizing agents such as peroxides, perborates, percarbonates, peroxyacids, persulfates, metal chlorites, and combinations thereof. Suitable peroxide compounds include hydrogen peroxide, urea peroxide, calcium peroxide, and mixtures thereof. An example of a percarbonate is sodium percarbonate. Other suitable whitening agents include potassium, ammonium, sodium and lithium persulfates and perborate mono- and tetrahydrates, and sodium pyrophosphate peroxyhydrate. Suitable metal chlorites include calcium chlorite, barium chlorite, magnesium chlorite, lithium chlorite, sodium chlorite, and potassium chlorite. In one embodiment chlorite is sodium chlorite. Additional whitening actives may be hypochlorite and chlorine dioxide.

Levels of whitening agents are generally from about 0.5% to about 15%, in another embodiment from about 1% to about 10%, by weight of the composition.

Vitamins and Minerals

The present invention may also comprise a safe and effective amount of vitamins or minerals. As used in this disclosure, the term vitamin refers to trace organic substances that are required in the diet. For the purposes of the present invention, the term vitamin(s) include, without limitation, thiamin, riboflavin, nicotinic acid, pantothenic acid, pyridoxine, biotin, folic acid, vitamin B 12, lipoic acid, ascorbic acid, vitamin A, vitamin D, vitamin E and vitamin K. Also included within the term vitamin are the coenzymes thereof. Coenzymes are specific chemical forms of vitamins. Coenzymes include thiamine pyrophosphates (TPP), flavin mononucleotide (FMM), flavin adenine dinucleotive (FAD), Nicotinamide adenine dinucleotide (NAD), Nicotinamide adenine dinucleotide phosphate (NADP) Coenzyme A (CoA) pyridoxal phosphate, biocytin, tetrahydrofolic acid, coenzyme B 12, lipoyllysine, 11-cis-retinal, and 1,25-dihydroxycholecalciferol. The term vitamin(s) also includes choline, camitine, and alpha, beta, and gamma carotenes.

As used in this disclosure, the term “mineral” refers to inorganic substances, metals, and the like required in the human diet. Thus, the term “mineral” as used herein includes, without limitation, calcium, iron, zinc, selenium, copper, iodine, magnesium, phosphorus, manganese, chromium and the like, and mixtures thereof.

Vitamins and minerals also include oral nutritional supplements such as amino acids, lipotropics, fish oil, and mixtures thereof, as disclosed in Drug Facts and Comparisons (loose leaf drug information service), Wolters Kluer Company, St. Louis, Mo., © 1997, pp. 54-54e. Amino acids include, but are not limited to L-Tryptophan, L-Lysine, Methionine, Threonine, Levocarnitine or L-carnitine and mixtures thereof. Lipotropics include, but are not limited to choline, inositol, betaine, linoleic acid, linolenic acids, and mixtures thereof. Fish oil contains large amounts of Omega-3 (N-3) Polyunsaturated fatty acids, eicosapentaenoic acid and docosahexaenoic acid.

As used with reference to a vitamin or mineral, the term “effective amount” means an amount at least about 10% of the United States Recommended Daily Allowance (“RDA”) of that particular ingredient for a patient. For example, if an intended ingredient is vitamin C, then an effective amount of vitamin C would include an amount of vitamin C sufficient to provide 10% or more of the RDA. Typically, where the tablet includes a mineral or vitamin, it will incorporate higher amounts, preferably about 100% or more of the applicable RDA.

### Chewable Solid Unit Dosage Form

The term “chewable solid unit dosage form” as used herein is meant a chewable tablet, capsule, hard and soft candy confections, toffee, nougat, chewy candy and the like. In one embodiment the chewable solid unit dosage forms are compressed tablets, soft gelatin capsules, molded tablets, molded sphere or ellipsoid made from any pharmaceutically acceptable excipient that can be melted or molded, gummy bear type forms, extruded solid forms, etc. In another embodiment the chewable solid unit dosage form is selected from the group consisting of compressed tablets or capsules. In one embodiment the chewable solid unit dosage form is a compressed tablet. The solid unit dosage form herein can also be a layered form, including one or more layers.

In another embodiment the unit dosage form is a compressed tablet of any shape or size, e.g. spherical or elliptical tablet. The tablet is compressed using conventional equipment and processes, for example see Lieberman, et al, Pharmaceutical Dosage Forms: Tablets (1980) Chapter 3, pp. 109-185. In one embodiment the unit dosage form of the present invention comprises a unit dosage form from about 100 mg to about 5 gram total weight, in another embodiment from about 250 mg to about 2 grams total weight, and in even another embodiment from about 500 mg to about 1.5 grams total weight.

The dosage form may also, in one embodiment, comprise an inert molded spherical or elliptical substrate. As used herein, “molding” refers to a process in which a molten or semi-solid inert, pharmaceutically acceptable material is injected into a mold cavity and allowed to solidify. The dimensions of the mold cavity thereby determine those of the substrate. Suitable materials include, but are not limited to, ingestible pharmaceutically acceptable waxes such as beeswax, paraffins, carnuba wax, and triglycerides with a melting point above about 50° C. such as tristearin. The active agent may be incorporated into the substrate during the molding process or coated onto molded substrates.

A still further preferred unit dosage form is a hard capsule (i.e. starch, cellulose, or gelatin hard capsules). The starch capsule may be filled with a solid form of active agent as described above. The preparation of the above tablets, capsules and hard and soft candy is well known in the art. In one embodiment for compressed dentifrice tablets, granulation of the dentifrice abrasive is necessary for the typically small particle sized abrasives used. Granulation is preferred for providing flow for subsequent processing and to impart compactibility on these materials. A wet granulation method can be used as follows:

a) Blend abrasive and sorbitol and/or mannitol (or other appropriate bulk filler).

b) Prepare binder solution by dissolving binding agent in water or other appropriate solvent.

c) Add binder solution b) to powder blend a) with appropriate mixing/agitation until properly wetted.

d) Optionally wet mill the material to break up large wet agglomerates.

e) Dry by appropriate means to an appropriate water/granulation solvent content (tray dry or, fluid bed dry, for example).

f) Optionally dry mill the dried granulation to yield appropriate particle size of the granulation.

Wet granulation can be accomplished by other processing means: for example; fluid bed granulation, wet mass extrusion, extrusion and spheronization, fluid bed roto-processing, and shugi processing.

In one embodiment granulation can also be accomplished by a dry granulation method as follows:

a) Blend abrasive and sorbitol and/or mannitol (or other appropriate compactible bulk filler).

b) Compact into large tablets (slugging press) or ribbons/brickets (roller compactor).

c) Dry mill product of b) to yield appropriate particle size of the granulation.

For both dry and wet granulation methods, other ingredients can be included in this step. For example, active agent may be added to the powder blend or binder solution to ensure proper content uniformity of the particular agent. Colorants, flavorants, surfactants, foaming agents, actives, etc. may also be added. In one embodiment the final blends for tabletting are prepared as follows:

a) Combine granulation from above with all other remaining components, except lubricant and blend appropriately to ensure uniformity.

b) Add lubricant and blend as needed.

Tabletting can be accomplished via traditional means for example one can compress the final powder blend from above on a tabletting press to form compacts of appropriate properties such as sufficient hardness and friability.

Alternatively, if a blend of the formula components have sufficient flow properties, and can form a reasonable compact, a direct compression method can be used whereby components are simply blended and tabletted without the need for a granulation step.

### Topical, Oral Care Carriers

In addition to the essential ingredients, the compositions of this invention also generally comprise topical, oral care carriers. As used herein, “topical, oral care carrier” or “oral carrier” means one or more compatible solid or liquid filler diluents or encapsulating substances which are suitable for administration to a subject or suitable for topical, oral administration. The term “compatible”, as used herein, means that the components of the composition are capable of being commingled with the active agent or other essential ingredients, and with each other, in a manner such that there is no interaction which would substantially reduce the efficacy of the composition under ordinary use situations. Oral care carriers must, of course, be of sufficiently high purity and sufficiently low toxicity to render them suitable for administration to the subject being treated. Oral care carriers may act to facilitate incorporation of the active agent into the dosage form, modify the release of the active agent from the dosage form, stabilize the active agent, or enhance absorption of the active agent. Oral care carriers should be safe for their intended use at the levels employed in the formulation. The formulation of active agent and oral care carriers is selected according to criteria well known to those skilled in the art to achieve the desired release rate, stability, absorption, and to facilitate the dosage form manufacture.

In one embodiment the oral care carrier is non-cariogenic and has low or no hygroscopic properties.

Oral care carriers generally include fillers or diluents, binders, disintegrating agents and lubricants. Fillers for example are generally selected from the group consisting of lactose, sucrose, dextrose, mannitol, sorbitol, xylitol, erythritol, lactitol, isomalt, maltitol, trehalose, tegatose, calcium sulfate, bibasic calcium phosphate, tricalcium phosphate, tribasic calcium sulfate, starch, such as cornstarch, potato starch, hydrogenated starch hydrolysates, and sodium starch glycolate, calcium carbonate, microcrystalline cellulose, and mixtures thereof. In one embodiment the filler is a noncariogenic polysaccharide, isomalt, and mixtures thereof. See, the above discussion regarding the use of cariogenic polysaccharides.

Lubricant, as used herein, means a material which can reduce the friction arising at the interface of the tablet and the die wall during compression and ejection thereof. Lubricants may also serve to prevent sticking to the punch and to the die wall. The term “antiadherents” is sometimes used to refer specifically to substances which function during ejection. As used in the present disclosure, however, the term “lubricant” is used generically and includes “antiadherents”.

Lubricants may be intrinsic or extrinsic. A lubricant which is directly applied to the tableting tool surface in the form of a film, as by spraying onto the die cavity and/or punch surfaces, is known as an extrinsic lubricant. Their use, however, requires complex application equipment and methods which add cost and reduce productivity. Intrinsic lubricants are incorporated in the material to be tableted. Traditional intrinsic lubricants include stearic acid, magnesium and calcium stearate, zinc stearate, hydrogenated and partially hydrogenated vegetable oils (e.g. peanut oil, cottonseed oil, sesame oil, olive oil, corn oil, and oil of theobroma, Sterotex), animal fats, glycerin, polyethylene glycol, polyoxyethylene monostearate, talc, light mineral oils, sodium benzoate, sodium lauryl sulphate, magnesium oxide and the like, and mixtures thereof. See European Patent Application No. 0,275,834, and Leal, et al., U.S. Pat. No. 3,042,531.

Intrinsic lubricants, according to the present invention, can optionally be used in an effective amount for example up to 5 weight percent and in another embodiment from about 0.25% to about 5%, in another embodiment from about 0.5% to about 2% by weight of the total composition.

Other topical, oral care carriers include emulsifiers, such as the Tweens®; wetting agents such as sodium lauryl sulfate; coloring agents; tableting agents; stabilizers; antioxidants; preservatives; pyrogen-free water; isotonic saline; and phosphate buffer solutions. Tablet carriers are described in Remington's Pharmaceutical Sciences, Mack Publishing Co. (19th edit. 1995); Modem Pharmaceutics, Vol. 7, Chapters 9 & 10, Banker & Rhodes (1979); Lieberman, et al, Pharmaceutical Dosage Forms: Tablets (1981); and Ansel, Introduction to Pharmaceutical Dosage Forms, 2d (1976). Their selection will depend on secondary considerations like taste, cost, and shelf stability, etc. and can be made without difficulty by those skilled in the art.

Other types of topical oral care carriers which may be included in compositions of the present invention, along with specific non-limiting examples, are:

Foaming Agent/Surfactant

The present composition may also contain suitable foaming agents such as those which are reasonably stable and form foam throughout a wide pH range. Foaming agents include nonionic, anionic, amphoteric, cationic, zwitterionic, synthetic detergents, and mixtures thereof. Many suitable nonionic and amphoteric surfactants are disclosed by U.S. Pat. No. 3,988,433 to Benedict; U.S. Pat. No. 4,051,234, issued Sep. 27, 1977, and many suitable nonionic surfactants are disclosed by Agricola et al., U.S. Pat. No. 3,959,458, issued May 25, 1976. In one embodiment the ratio of retentive agent to surfactant is greater than about 1, in another embodiment is greater than about 2, and in yet another embodiment is greater than about 3.

The present composition optionally comprises a safe and effective amount of a foaming agent, in another embodiment comprises from about 0.001% to about 20%, in another embodiment from about 0.05% to about 9%, and in even another embodiment from about 0.1% to about 5% by weight of the composition of foaming agent. In one embodiment the foaming agent is selected from the group consisting of cocamidopropyl betaine, sodium alkyl sulfate, poloxamer, PEG-40 sorbitan isostearate, and mixtures thereof.

Anionic Surfactants

Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate and sodium coconut monoglyceride sulfonates are examples of anionic surfactants of this type. Other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Mixtures of anionic surfactants can also be employed.

Abrasive

The present composition may also optionally include a dental abrasive. Dental abrasives useful in the compositions of the subject invention include many different materials. The material selected must be one which is compatible within the composition and does not excessively abrade dentin. Suitable abrasives include, for example, silicas including gels and precipitates, insoluble sodium polymetaphosphate, hydrated alumina, calcium carbonate, dicalcium orthophosphate dihydrate, calcium pyrophosphate, tricalcium phosphate, calcium polymetaphosphate, and resinous abrasive materials such as particulate condensation products of urea and formaldehyde, and mixtures thereof.

The level of optional abrasive in the compositions described herein is generally from about 6% to about 70% by weight of the composition, in another embodiment is from about 10% to about 60% of abrasive, in another embodiment from about 15% to about 50%, and in yet another embodiment from about 15% to about 40%, by weight of the composition.

In one embodiment the level of water insoluble particulates of the present invention (e.g. some abrasives, fillers, etc.) are less than about 65%, in another embodiment less than about 60%, in another embodiment less than about 50%, by weight of the composition.

Another class of abrasives for use in the present compositions is the particulate thermo-setting polymerized resins as described in U.S. Pat. No. 3,070,510 issued to Cooley & Grabenstetter on Dec. 25, 1962. Suitable resins include, for example, melamines, phenolics, ureas, melamine-ureas, melamine-formaldehydes, urea-formaldehyde, melamine-urea-formaldehydes, cross-linked epoxides, and cross-linked polyesters.

Silica dental abrasives of various types are preferred because of their unique benefits of exceptional dental cleaning and polishing performance without unduly abrading tooth enamel or dentine. The silica abrasive polishing materials herein, as well as other abrasives, generally have an average particle size ranging between about 0.1 to about 30 microns, and preferably from about 5 to about 15 microns. The abrasive can be precipitated silica or silica gels such as the silica xerogels described in Pader et al., U.S. Pat. No. 3,538,230, issued Mar. 2, 1970, and DiGiulio, U.S. Pat. No. 3,862,307, issued Jan. 21, 1975. in one embodiment the silica abrasives are the silica xerogels marketed under the trade name “Syloid” by the W.R. Grace & Company, Davison Chemical Division. In another embodiment the silica abrasives are the precipitated silica materials such as those marketed by the J. M. Huber Corporation under the trade name, Zeodent®, particularly the silica carrying the designation Zeodent 119®. The types of silica dental abrasives useful in solid unit dosage forms that are toothpastes are described in more detail in Wason, U.S. Pat. No. 4,340,583, issued Jul. 29, 1982.

A particularly preferred precipitated silica is the silica disclosed in U.S. Pat. No. 5,603,920, issued on Feb. 18, 1997; U.S. Pat. No. 5,589,160, issued Dec. 31, 1996; U.S. Pat. No. 5,658,553, issued Aug. 19, 1997; U.S. Pat. No. 5,651,958, issued Jul. 29, 1997, all of which are assigned to the Procter & Gamble Co.

Mixtures of abrasives may be used.

Flavoring and Sweetening Agents

Flavoring agents may also optionally be added to the compositions. Suitable flavoring agents include oil of wintergreen, oil of peppermint, oil of spearmint, clove bud oil, menthol, anethole, methyl salicylate, eucalyptol, 1-menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal known as CGA, and mixtures thereof. Flavoring agents are generally used in the compositions at levels of from about 0.001% to about 5%, by weight of the composition.

Sweetening agents which can be optionally used include sucralose, sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame and cyclamate salts, especially sodium cyclamate and sodium saccharin, and mixtures thereof. In one embodiment the composition comprises from about 0.1% to about 10% of these agents, in another embodiment from about 0.1% to about 1%, by weight of the composition.

In addition to flavoring and sweetening agents, coolants, salivating agents, warming agents, and numbing agents can be used as optional ingredients in compositions of the present invention. These agents are present in the compositions at a level of from about 0.001% to about 10%, in another embodiment from about 0.1% to about 1%, by weight of the composition.

The coolant can be any of a wide variety of materials. Included among such materials are carboxamides, menthol, ketals, diols, and mixtures thereof. Preferred coolants in the present compositions are the paramenthan carboxyamide agents such as N-ethyl-p-menthan-3-carboxamide, known commercially as “WS-3”, N,2,3-trimethyl-2-isopropylbutanamide, known as “WS-23,” and mixtures thereof. Additional coolants may be selected from the group consisting of menthol, 3-1-menthoxypropane-1,2-diol known as TK-10 manufactured by Takasago, menthone glycerol acetal known as MGA manufactured by Haarmann and Reimer, and menthyl lactate known as Frescolat® manufactured by Haarmann and Reimer. The terms menthol and menthyl as used herein include dextro- and levorotatory isomers of these compounds and racemic mixtures thereof. TK-10 is described in U.S. Pat. No. 4,459,425, Amano et al., issued Jul. 10, 1984. WS-3 and other agents are described in U.S. Pat. No. 4,136,163, Watson, et al., issued Jan. 23, 1979.

Salivating agents of the present invention include Jambu® manufactured by Takasago. Warming agents include capsicum and nicotinate esters, such as benzyl nicotinate. Numbing agents include benzocaine, lidocaine, clove bud oil, and ethanol. Mixtures of these agent may be used.

Sensitivity Agents/Anesthetic Agents

Anti-pain or desensitizing agents may also optionally be present in the compositions of the present invention. Analgesics are agents that relieve pain by acting centrally to elevate pain threshold without disturbing consciousness or altering other sensory modalities. Such agents may include, but are not limited to, strontium chloride, potassium nitrate, sodium nitrate, sodium fluoride, acetanilide, phenacetin, acertophan, thiorphan, spiradoline, aspirin, codeine, thebaine, levorphenol, hydromorphone, oxymorphone, phenazocine, fentanyl, buprenorphine, butaphanol, nalbuphine, pentazocine, natural herbs such as gall nut, Asarum, Cubebin, Galanga, scutellaria, Liangmianzhen, Baizhi, etc. Anesthetic agents, or topical analgesics, such as acetaminophen, sodium salicylate, trolamine salicylate, lidocaine and benzocaine may also be present. These analgesic actives are described in detail in Kirk-Othmer, Encyclopedia of Chemical Technology, Fourth Edition, Volume 2, Wiley-Interscience Publishers (1992), pp. 729-737.

Miscellaneous Oral Care Carriers

The chewable solid unit dosage forms of the present invention, in one embodiment have less than about 5% disintegrants, in another embodiment have less than about 3% disintegrants, and in another embodiment have less than about 1% or are essentially free of disintegrants.

### Composition Use

The present compositions can be used at home by the consumer. The present compositions are used, in one embodiment, from about once per week to about four times per day, in another embodiment from about thrice per week to about three times per day, in even another embodiment from about once per day to about twice per day. The period of such treatment typically ranges from about one day to a lifetime. For particular oral care diseases or conditions the duration of treatment depends on the severity of the oral disease or condition being treated, the particular delivery form utilized and the patient's response to treatment. In one embodiment the duration of treatment is from about 3 weeks to about 3 months, but may be shorter or longer depending on the severity of the condition being treated, the particular delivery form utilized and the patient's response to treatment.

The present invention further relates to a method of providing sustained delivery of an oral care active, in the oral cavity of a subject in need thereof, for the treatment or prevention of an oral condition alone or for promoting whole body health, by administering topically, an oral care composition comprising:

a. from about 1% to about 40%, by weight of the composition, of a retentive agent selected from the group consisting of water soluble hydrophilic gums, water soluble hydrophilic polymers, and mixtures thereof, the retentive agent having the property of hydrating upon exposure to water or saliva, in one embodiment resulting in the composition forming an intact hydrated mass to provide a Retention Index of about 1 to about 4; and b. a safe and effective amount of a topical, oral care carrier; wherein the composition is a non-cariogenic, chewable solid unit dosage form; and the composition comprises less than about 65% by weight of water insoluble particulates.

The present invention further relates to a method of providing sustained delivery of an oral care active, in the oral cavity of a subject in need thereof, for the treatment or prevention of an oral condition alone or for promoting whole body health, by administering topically, an oral care dentifrice composition comprising: a. from about 30% to about 65%, by weight of the composition, of a water insoluble, particulate retentive agent having a water solubility of less than about 1 g/30 g at 25° C.; b. a safe and effective amount of an oral care active; c. a safe and effective amount of a surfactant; d. a safe and effective amount of an oral care carrier selected from the group consisting of a flavor, sensate, buffer, and mixtures thereof; wherein the composition is a chewable solid unit dosage for, non-effervescent, non-cariogenic; and wherein, in one embodiment the composition has a Retention Index of from about 1 to about 4.

The present invention further relates to a method of providing sustained delivery of a flavor, sensate or buffer, in the oral cavity of a subject in need thereof, by administering topically, an oral care composition comprising: a. from about 1% to about 40%, by weight of the composition, of a retentive agent selected from the group consisting of water soluble hydrophilic gums, water soluble hydrophilic polymers, and mixtures thereof, the retentive agent having the property of hydrating upon exposure to water or saliva in one embodiment resulting in the composition forming an intact hydrated mass to provide a Retention Index of about 1 to about 4; and b. a safe and effective amount of a topical, oral care carrier selected from the group consisting of a flavor, sensate, buffer, and mixtures thereof; wherein the composition is a non-cariogenic, chewable solid unit dosage form; and the composition comprises less than about 65% by weight of water insoluble particulates.

The present invention further relates to a method of providing sustained delivery of a flavor, sensate or buffer, in the oral cavity of a subject in need thereof, by administering topically, an oral care dentifrice composition comprising: a. from about 30% to about 65%, by weight of the composition, of a water insoluble, particulate retentive agent having a water solubility of less than about 1 g/30 g at 25° C.; b. a safe and effective amount of an oral care active; c. a safe and effective amount of a surfactant; d. a safe and effective amount of an oral care carrier selected from the group consisting of a flavor, sensate, buffer, and mixtures thereof; wherein the composition is a chewable solid unit dosage for, non-effervescent, non-cariogenic; and wherein in one embodiment the composition has a Retention Index of from about 1 to about 4.

The compositions of this invention are useful for both human and other animal (e.g. pets, zoo, or domestic animals) applications.

### EXAMPLES

The following non-limiting examples further describe preferred embodiments within the scope of the present invention. Many variations of these examples are possible without departing from the scope of the invention.

### Example I

The following chewable compressed tablets, containing sodium fluoride, are made by conventional tableting processing techniques by mixing the following:

| | #1 | #2 | #3 | #4 | #5 |
|---|---|---|---|---|---|
| Material | % w/w | % w/w | % w/w | % w/w | % w/w |
| Na Fluoride | 0.243 | 0.0884 | 0.0552 | 0.11 | 0.11 |
| Na Lauryl Sulfate | 1.5 | | | 1.5 | 1.5 |
| Poloxamer 407 | | 7.5 | | | |
| PEG 40 Sorbitan Di-iso Stearate | | | 2 | | |
| Silica | 20 | | | 20 | 20 |
| Ca Pyrophosphate | | 40 | | | |
| Dicalcium Phosphate | | | 40 | | |
| Tetra Sodium Pyrophosphate | | 5 | 5 | | 5 |
| Na Saccharin | 0.5 | | 0.4 | 0.4 | 0.4 |
| Acesulfame K | | .3 | | | |
| Sucralose | | | 0.1 | | |
| Aspartame | | .3 | | | |
| Flavor | 1.5 | 1.5 | 1.5 | 1.5 | 1.5 |
| Na Bicarbonate | | 5 | | 5 | 10 |
| Dibasic | | | 5 | | |
| Na Phosphate | | | | | |
| Methocel K4M Premium | | 10 | 5 | | |
| (Hydroxypropylmethyl Cellulose) | | | | | |
| Methocel K100LV Premium | | | 10 | | |
| (Hydroxypropylmethyl Cellulose) | | | | | |
| Na Carboxymethylcellulose (7H3 | | | | 6 | 15 |
| Aqualon) | | | | | |
| Hydroxyethyl Cellulose (Klucel 250 | | | | 3 | |
| M Aqualon) | | | | | |
| Xanthan Gum | 2 | | | | |
| Microcrystaline Cellulose | 5 | 10 | 5 | | |
| Polyvinyl Pyrrolidone | 3 | | 3 | | |
| Croslinked Na Carboxymethyl | 2 | | | | |
| Cellulose | | | | | |
| Croslinked Polyvinyl Pyrrolidone¹ | | 2 | 2 | | |
| Sorbitol | 30 | 16.8116 | 19.4448 | 33 | 23 |
| Mannitol | 33.257 | 0 | 0 | 28.49 | 22.49 |
| Cetyl pyrridinium Chloride | | 0.5 | | | |
| Chlorhexidine Gluconate | | | 0.5 | | |
| Zinc Stearate | 1 | 1 | 1 | 1 | 1 |
| Total | 100 | 100 | 100 | 100 | 100 |

### Example II

The following chewable compressed tablets, containing sodium monofluorophosphate, are made by conventional tableting processing techniques by mixing the following:

| | #1 | #2 |
|---|---|---|
| Material | % w/w | % w/w |
| Sodium Monofluorophosphate | 0.833 | 0.150 |
| Na Lauryl Sulfate | 1.5 | |
| PEG 40 Sorbitan Di-iso Stearate | | 2 |
| Silica | 20 | |
| Dicalcium Phosphate | | 40 |
| Tetra Na Pyrophosphate | 5 | |
| Na Saccharin | 0.5 | 0.5 |
| Flavor | 1.5 | 1.5 |
| Na Bicarbonate | 10 | |
| Dibasic | | 5 |
| Na Phosphate | | |
| Methocel K4M Premium | | 4 |
| (Hydroxypropylmethyl Cellulose) | | |
| Methocel K100LV Premium | | 8 |
| (Hydroxypropylmethyl Cellulose) | | |
| Na Carboxymethyl Cellulose (Cekol 30000) | 7 | |
| Polymethyl vinyl ether/maleic anhydride (Ca/Zn | 12 | |
| Salt) | | |
| Microcrystalline Cellulose | 5 | |
| Polyvinyl Pyrrolidone | 3 | 3 |
| Croslinked Polyvinyl Pyrrolidone² | 1 | 0 |
| Sorbitol | 15 | 20 |
| Mannitol | 14.667 | 14.35 |
| Zinc Chloride | 2.5 | |
| Copper Chloride | | 0.5 |
| Zinc Stearate | 0.5 | 1.0 |
| Total | 100 | 100 |

¹Plasdone XL from ISP.

### Example III

The following chewable compressed tablets, containing stannous fluoride, are made by conventional processing techniques by mixing the following:

| | #1 | #2 |
|---|---|---|
| Material | % w/w | % w/w |
| Stannous Fluoride | 0.454 | 0.0825 |
| Na Lauryl Sulfate | 1.5 | |
| PEG 40 Sorbitan Di-iso Stearate | | 2 |
| Silica | 20 | 10 |
| Aluminum Oxide | | 5 |
| Na Polyphosphate (Glass H)³ | 7 | 7 |
| Na Saccharin | 0.5 | 0.5 |
| Flavor | 1.5 | 1.5 |
| Na Bicarbonate | 10 | |
| Dibasic | | 5 |
| Na Phosphate | | |
| Methocel K4M Premium | 5 | 7.5 |
| (Hydroxypropylmethyl Cellulose) | | |
| Methocel K100LV Premium | 10 | 7.5 |
| (Hydroxypropylmethyl Cellulose) | | |
| Microcrystalline Cellulose | 5 | 0 |
| Polyvinyl Pyrrolidone | 3.0 | 0 |
| Croslinked Polyvinyl Pyrrolidone⁴ | 2 | 2 |
| Sorbitol | 12.046 | 20 |
| Mannitol | 20 | 30.9175 |
| Zinc Chloride | 1 | |
| Zinc Stearate | 1 | 1 |
| Total | 100 | 100 |

³n = 21 from FMC.
⁴Plasdone XL from ISP.

### Example IV

The following chewable compressed tablets, having no fluoride ion source, are made by conventional tableting processing techniques by mixing the following:

| | #1 | #2 | #3 |
|---|---|---|---|
| Material | (% w/w) | (% w/w) | (% w/w) |
| Na Lauryl Sulfate | 1.5 | | 1.5 |
| Cocamidopropyl Betaine | | 2 | |
| Silica | 20 | | |
| Calcium Carbonate | | 40 | |
| Di Calcium Phosphate | | | 40 |
| Tetra Na Pyrophophate | 5 | | |
| Na Tripolyphosphate | | 7 | |
| Na Polyphosphate (Glass H)⁵ | | | 10 |
| Na Saccharin | .5 | .5 | .5 |
| Flavor | 1.5 | 1.5 | 1.5 |
| Na Bicarbonate | 10 | 5 | 7 |
| Methocel K4M Premium | 5 | 6 | |
| (Hydroxypropylmethyl Cellulose) | | | |
| Methocel K100LV Premium | 10 | 6 | |
| (Hydroxypropylmethyl Cellulose) | | | |
| Na Alginate (Protanol LF 200s) | | | 10 |
| Microcrystalline Cellulose | 5 | | |
| Polyvinyl Pyrrolidone | 1.2 | 1.2 | |
| Na Starch Glycolate | | 2 | |
| Sorbitol | 20 | 27.55 | |
| Mannitol | 19.02 | | 27.5 |
| Triclosan | 0.28 | | |
| Cetylpyrridinium Chloride | | 0.25 | 1 |
| Zinc Stearate | 1 | 1 | 1 |
| Total | 100 | 100 | 100 |

⁴Plasdone XL from ISP
⁵n = 21 from FMC.

### Example V

The following chewable compressed tablets, are made by conventional tableting processing techniques by mixing the following:

| | #1 | #2 | #3 |
|---|---|---|---|
| Material | (% w/w) | (% w/w) | (% w/w) |
| Sorbitol, NF (D-glucitol) | | | 15.000 |
| Calcium Carbonate | 46.875 | 37.965 | |
| Sodium Fluoride | 0.088 | 0.177 | 0.324 |
| Isomalt (hydrogenated isomaltulose) | | | 32.401 |
| Mannitol, USP | 39.837 | 34.250 | |
| Magnesium Aluminum Silicate | | | 45.000 |
| Hydroxypropylmethyl Cellulose | | | 3.150 |
| Polyvinyl Pyrrolidone | 4.099 | 3.308 | |
| Hydroxyethylcellulose | | 2.000 | |
| Carboxymethyl Cellulose | | 5.000 | |
| Sodium Alkyl Sulfate Powder | | 1.500 | 0.875 |
| Cocamidopropyl Betaine | 1.750 | | |
| Sodium Bicarbonate | | 10.000 | |
| Sodium Saccharin, USP | 1.000 | 1.100 | 0.850 |
| Flavor | 1.500 | 1.250 | 1.600 |
| Talc | 2.501 | 1.950 | |
| Magnesium Stearate | 2.350 | 1.500 | 0.800 |
| Total | 100.000 | 100.000 | 100.000 |

While particular embodiments of the present invention have been described, it will be obvious to those skilled in the art that various changes and modifications of the present invention can be made without departing from the spirit and scope of the invention. It is intended to cover, in the appended claims, all such modifications that are within the scope of this invention.

What is claimed is:

1. An oral care dentifrice composition comprising:
a. from about 30% to about 65%, by weight of the composition, of a water insoluble, particulate retentive agent having a water solubility of less than about 1 g/30 g at 25° C.;
b. a safe and effective amount of an oral care active;
c. a safe and effective amount of a surfactant;
d. a safe and effective amount of a buffer;
wherein the composition is a chewable dentifrice solid unit dosage form, is non-effervescent, non-cariogenic; and wherein the composition has a Retention Index of from about 1 to about 4.

2. The composition of claim 1 wherein the retentive agent has a water solubility of less than about 1 g/100 g at 25° C.

3. The composition of claim 1 wherein the Retention Index is from about 2 to about 4.

4. The composition of claim 2 wherein from about 0.5% to about 20% by weight of the initial composition deposits in some of the tooth surfaces after chewing by the subject.

5. The composition of claim 1 wherein the retentive agent is at a level of from about 30% to about 60%, by weight of the composition.

6. The composition of claim 5 wherein the retentive agent is selected from the group consisting of calcium carbonate, mica, titanated mica, magnesium carbonate, talc (magnesium silicate), magnesium aluminum silicate, kaolin (aluminum silicate), titanium dioxide, zinc oxide, polyethylene powder, polystyrene powder, bismuth oxychloride, and mixtures thereof.

7. The composition of claim 6 wherein retentive agent is selected from the group consisting of calcium carbonate, magnesium carbonate, talc (magnesium silicate), magnesium aluminum silicate, and mixtures thereof.

8. The composition of claim 1 wherein the oral care active agent is selected from the group consisting of anticalculus agent, fluoride ion source, antimicrobial agents, dentinal desensitizing agents, anesthetic agents, antifungal agents, anti-inflammatory agents, selective H-2 antagonists, anticaries agents, remineralization agents, whitening agents, antierosion agents, vitamins, minerals, and mixtures thereof.

9. The composition of claim 8 wherein the active agent is a fluoride ion source providing from about 200 ppm to about 300 ppm of fluoride ion.

10. The composition of claim 1 wherein the solid unit dosage form is a compressed tablet.

11. The composition of claim 1 wherein the buffer is selected from the group consisting of water soluble buffers, sodium bicarbonate, sodium carbonate, phosphate buffers, amino acid buffers, alanine, glycine, trisodium phosphate, disodium phosphate, disodium hydrogen phosphate, sodium dihydrogen phosphate, tris(hydroxymethyl) aminomethane, tetrasodium pyrophosphate, disodium pyrophosphate; tetrapotassium pyrophosphate, salts of tripolyphosphate, and mixtures thereof.

12. An oral care kit comprising:
a. an oral care dentifrice composition for topical, oral administration in a human or other animal comprising:
1. from about 30% to about 65%, by weight of the composition, of a water insoluble, particulate retentive agent having a water solubility of less than about 1 g/30 g at 25° C.; and
2. a safe and effective amount of an oral care active;
3. a safe and effective amount of surfactant;
4. a safe and effective amount of buffer;
b. instructions for use to chew the composition and thereafter brush the teeth; and
c. a container;
wherein the composition is a chewable dentifrice solid unit dosage form, is non-effervescent, and non-cariogenic.

13. A method of buffering the oral cavity saliva or environment on or at the tooth surfaces of a subject in need thereof, to a pH from about 7 to about 12, for at least about 2 minutes, by administering topically to the oral cavity, an oral care composition, the composition comprising:
a. from about 30% to about 65%, by weight of the composition, of a water insoluble, particulate retentive agent having a water solubility of less than about 1 g/30 g at 25° C.;
b. a safe and effective amount of an oral care active;
c. a safe and effective amount of a surfactant;
d. a safe and effective amount of a buffer;
wherein the composition is a chewable dentifrice solid unit dosage form, non-effervescent, non-cariogenic and wherein the composition has a Retention Index of from about 1 to about 4.

14. A method of providing sustained delivery of an oral care active, in the oral cavity of a subject in need thereof, for treatment or prevention of an oral condition alone or for promoting whole body health, by administering topically, an oral care dentifrice composition. comprising:
a. from about 30% to about 65%, by weight of the composition, of a water insoluble, particulate retentive agent having a water solubility of less than about 1 g/30 g at 25° C.;
b. a safe and effective amount of an oral care active;
c. a safe and effective amount of a surfactant;
d. a safe and effective amount of an oral care carrier selected from the group consisting of a flavor sensate, buffer or mixtures thereof;
wherein the composition is a chewable solid unit dosage form, non-effervescent, and non-cariogenic.

15. The method of claim 13 wherein the retentive agent has a water solubility of less than about 1 g/100 g at 25° C.

16. A method of providing sustained delivery of a flavor, sensate or buffer, in the oral cavity of a subject in need thereof, by administering topically, an oral care dentifrice composition comprising:
a. from about 30% to about 65%, by weight of the composition, of a water insoluble, particulate retentive agent having a water solubility of less than about 1 g/30 g at 25° C.
b. a safe and effective amount of an oral care active;
c. a safe and effective amount of a surfactant;
d. a safe and effective amount of an oral care carrier selected from the group consisting of a flvor, sensate, buffer, and mixtures thereof;
wherein the composition is a chewable solid unit dosage form, non-effervescent, and non cariogenic.

17. The method of claim 16 wherein the retentive agent has a water solubility of less than about 1 g/100 g at 25° C.
